(19) United States
(12) Patent Application Publication
(10) Pub. No.: US 2019/0201710 A1
(43) Pub. Date: Jul. 4, 2019

(54) SYSTEM, DEVICE, AND METHOD FOR CROSS-LINKING CORNEAL TISSUE

(71) Applicant: Yichieh SHIUEY, San Jose, CA (US)

(72) Inventor: Yichieh SHIUEY, San Jose, CA (US)

(73) Assignee: Keramed, Inc., Fairfield, NJ (US)

(21) Appl. No.: 16/329,998

(22) PCT Filed: Sep. 27, 2016
(86) PCT No.: PCT/US2016/053849

§ 371 Date: Mar. 1, 2019

(51) Int. Cl. A61N 5/06

(52) U.S. Cl. CPC A61N 5/062; A61N 2005/0661; A61N 2005/0648

(57) ABSTRACT

System, device and method for cross-linking corneal tissue by inserting a membrane into corneal tissue and activating a radiation emitting component to effect cross-linking in desired areas within the cornea.

### BACKGROUND

Ultraviolet radiation can be used to cross-link conical collagen fibrils in corneas suffering from ectasia or other degenerative conditions, such as keratoconus, Pellucid Marginal Degeneration, Terrien Marginal Degeneration, and post-refractive surgery. Corneal cross-linking (“CXL”) strengthens the collagen, essentially through the formation of strong chemical bonds between adjacent fibrils, resulting in stiffer corneas that are less susceptible to degeneration.

Typically, a CXL procedure involves the application of a photosensitizer agent (e.g., a riboflavin solution) to the surface of the eye, followed by UV radiation treatment. The photosensitizer agent is excited by the radiation and then converts the absorbed energy partially into chemical energy to enhance chemical bonding of collagen fibrils, e.g., by forming cross-link bonds between amino acids in the tissue. The photosensitizer can be applied to a deepithelized cornea for enhanced and more efficient diffusion of the vitamin into the corneal tissue or alternatively to a cornea having its epithelium intact.

Typical CXL procedures suffer from a number of drawbacks. For example, it is virtually impossible to adequately control the precise depth of radiation penetration. This can result in insufficient tissue cross-linking and/or radiation damage to the deeper layers of the cornea and the eye, particularly when the cornea is relatively thin, which is frequently the case in patients who could benefit from a CXL procedure. Imprecision of radiation application to specific layers or areas of the cornea also significantly limits the types of procedures that might otherwise benefit from employing CXL. For example, CXL procedures lack the precision and controllability that are required to effect a refractive correction in the eye. Another drawback is the need, in most cases, to remove the epithelium of the patient's eye to provide sufficient photosensitizer diffusion, which is an extremely delicate procedure that can result in severe pain and discomfort, and can lead to post-surgical complications and disease. Leaving the epithelium intact results in a much longer procedure, as diffusion of the photosensitizer into the corneal tissue takes much longer than in a deepithelized cornea; and even then sufficient diffusion may not be attainable.

There is a need for improved CXL devices and methods.

### SUMMARY

In one aspect, the present disclosure is directed to a device for performing cross-linking of conical tissue, the device comprising a membrane and a radiation emitting component, the device being configured to be removably embedded in a cornea.

In another aspect, the present disclosure is directed to a system for performing cross-linking of conical tissue, the system comprising a reversibly deformable membrane, a radiation generator, and a radiation emitting component, the reversibly deformable membrane being configured to be removably embedded in a cornea.

In yet a further aspect, the present disclosure is directed to a method for performing cross-linking of corneal tissue, the method comprising the steps of: making a pocket in a cornea; introducing a photosensitizer into at least a portion of the cornea, such as the surface or interior of the cornea; placing a device in the pocket, the device comprising a reversibly deformable membrane; and activating the radiation emitting component to emit radiation, the radiation emitting component being selected to emit radiation that reacts with the photo sensitizer.

In still a further aspect, the present disclosure is directed to a system for performing cross-linking of corneal tissue, the system comprising a device being configured for removable embedding into corneal tissue and comprising a membrane and a plurality of radiation emitting components coupled to the membrane, the system further comprising a controller, the controller being configured to selectively activate the plurality of radiation emitting components while the device is embedded in the corneal tissue.

### BRIEF DESCRIPTION OF THE DRAWINGS

FIG. 1 is a schematic perspective view of an example system for performing cross-linking of corneal tissue in accordance with the present disclosure, including a schematic top view of an example device for performing cross-linking of corneal tissue, the device being shown in a first configuration.

FIG. 2 is a schematic perspective view of the system of FIG. 1, including a perspective view of the device of FIG. 1, the device being shown in the first configuration.

FIG. 3 is a schematic perspective view of the system of FIG. 1, including a perspective view of the device of FIG. 1, the device being shown in a second configuration.

FIG. 4 is a schematic perspective view of the system of FIG. 1, including the device of FIG. 1, the device being shown in a third configuration.

FIG. 5 is a schematic side view of a portion of a human eye showing a corneal pocket.

FIG. 6 is a schematic top view of the portion of the human eye of FIG. 5.

FIG. 7 is a schematic perspective view of the conical cross-linking device of FIG. 1 disposed in an implantation device for embedding the device in a pocket formed in the cornea of an eye.

FIG. 8 is a schematic perspective view of the system of FIG. 1, the device of FIG. 1 being disposed in a corneal pocket.

FIG. 9 is a schematic cross-sectional view of the device of FIG. 1 disposed in a corneal pocket.

FIG. 10A is a further example of a device for performing cross-linking of conical tissue in accordance with the present disclosure.

FIG. 10B is yet a further example of a device for performing cross-linking of conical tissue in accordance with the present disclosure.

FIG. 10C is yet a further example of a device for performing cross-linking of conical tissue in accordance with the present disclosure.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims. The drawings are not necessarily drawn to scale, nor is the scale of one drawing necessarily consistent with that of another drawing.

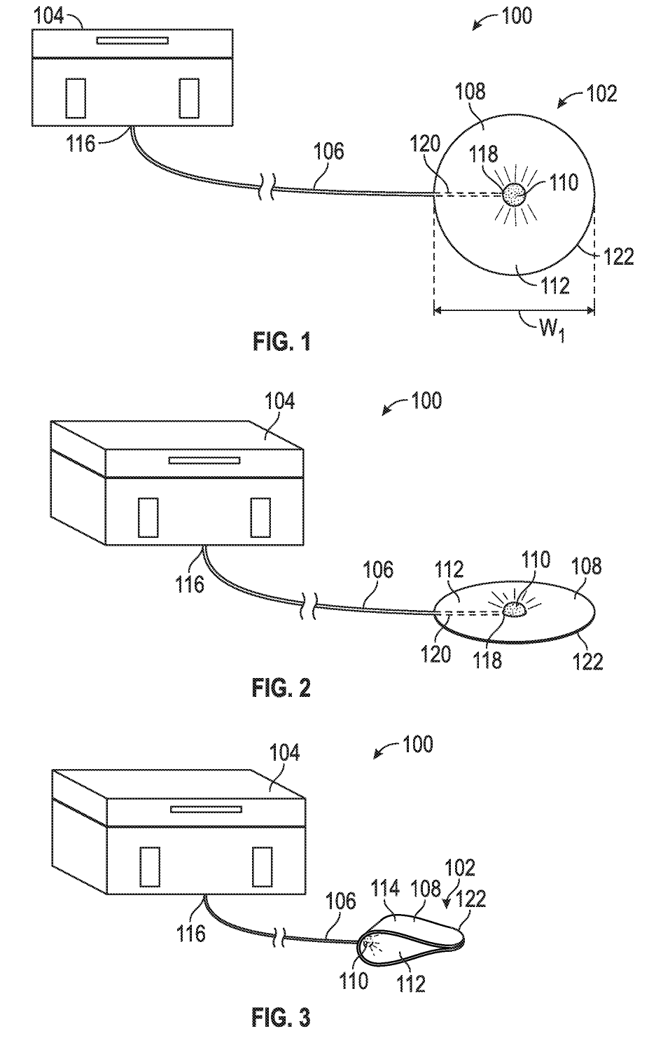

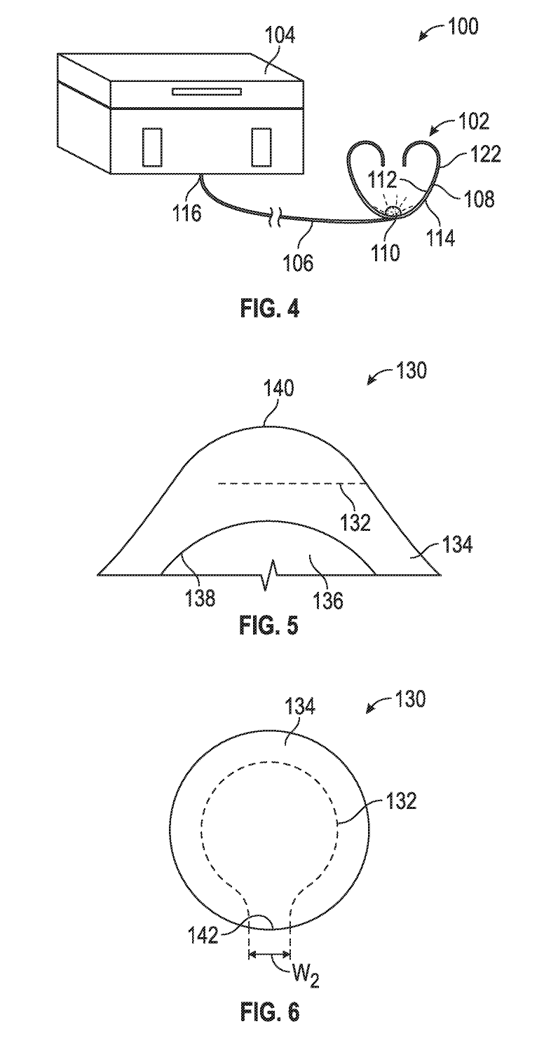

FIG. 1 is a schematic perspective view of an example system 100 for performing cross-linking of corneal tissue in accordance with the present disclosure, including a schematic top view of an example device 102 for performing cross-linking of corneal tissue, the device 102 being shown in a first configuration. FIG. 2 is a schematic perspective view of the system 100 of FIG. 1, including a perspective view of the device 102 of FIG. 1, the device 102 being shown in the first configuration. FIG. 3 is a schematic perspective view of the system 100 of FIG. 1, including a perspective view of the device 102 of FIG. 1, the device 102 being shown in a second configuration. FIG. 4 is a schematic perspective view of the system 100 of FIG. 1, including the device 102 of FIG. 1, the device 102 being shown in a third configuration.

With reference to FIGS. 1-4, the system 100 includes the device 102, a radiation generator 104 and a conduit 106. The device 102 includes a membrane 108 and a radiation emitting component 110. In some examples, the device 102 is reversibly deformable. In these examples, one or more components of the device 102 (e.g., the membrane 108 and/or the radiation emitting component 110), or portions thereof, is/are reversibly deformable. In addition, in some examples one or more portions of the conduit 106 is/are reversibly deformable. It should also be appreciated that the device 102 can be manufactured and/or provided in the deformed configuration, and then un-deformed or partially un-deformed by the practitioner when the device is implanted in a conical pocket.

The membrane 108 has a front surface 112 and a rear surface 114, the front surface 112 and the rear surface 114 defining a thickness there between. In some examples this thickness can be in a range from about 10 microns to about 500 microns. Thicknesses outside of this range may also be suitable.

The radiation generator 104 includes a power source that powers a signal generating module. The conduit 106 connects at one end to the radiation generator 104 and at an opposing end to the radiation emitting component 110. Signals generated by the signal generating module travel down the conduit 106 from the radiation generator 104 to the radiation emitting component 110 to thereby activate the radiation emitting component 110, i.e., to cause the radiation emitting component to emit radiation. In some examples the conduit 106 includes one or more optical fibers that transmit optical signals generated by the signal generating module to the radiation emitting component.

The radiation emitting component 110 can be any suitable radiation source, e.g., one or more light emitting diodes (LED). The radiation emitting component 110 can include one or more radiation emitting elements, e.g., LEDs. The radiation emitting component 110 can be configured to emit one or more wavelengths or ranges of wavelengths of electromagnetic radiation, such as ultraviolet light, visible light, and infrared light. In some examples, the radiation emitting component 110 is configured to emit ultraviolet (UV) light at a wavelength or wavelengths within the absorption spectrum for a photosensitizer agent (e.g., riboflavin) diffused in a cornea, such that exposure of the photosensitizer agent to the radiation results in cross-linking of collagen fibrils in the cornea.

In some examples, the radiation generator 104 can include a controller (e.g., integral to the radiation generator, or connected thereto) for controlling the characteristics of the radiation emitted by the radiation emitting component 110, including, e.g., the radiation's wavelength and/or power (as functions of time, and/or as functions of radiation emission direction and/or as functions of the radiation emission locations with respect to the front surface 112 of the membrane 108). For example, the radiation can be emitted from one or more LEDs located at different locations relative to the front surface 112, the LEDs emitting constant or non-constant (e.g., pulsing) radiation at different wavelengths and/or different powers from the various locations on the membrane 108. In some examples, one or more of the radiation generator 104, the conduit 106, and the radiation emitting component 110 is provided by a MIGHTEX High Power Fiber-Coupled LED Light Source.

The conduit 106 connects at a first end 116 to the radiation generator 104, and at a second end 118 to the radiation emitting component 110. In some examples, a portion 120 of the conduit 106 passes within the membrane 108, that is, a portion 120 of the conduit 106 is embedded in the membrane 108. In alternative examples, a portion of the conduit 106 is secured (e.g., with glue, heat adhesion, soldering, etc.) to an exterior surface (e.g., the front surface 112 or the rear surface 114) of the membrane 108. In alternative examples the conduit 106 is not secured to the membrane 108 and passes directly to the radiation emitting component 110. In some examples at least a portion of the conduit 106 that is adjacent the second end 118 has a thickness configured for insertion into conical tissue, e.g., a maximum thickness from 100 microns to 5 mm. Thicknesses outside of this range may also be suitable.

The conduit 106 can be flexible (e.g., bendable) or rigid. The conduit 106 is preferably configured to transmit signals (e.g., optical signals, electrical signals) that generate a desired wavelength or wavelengths of radiation emitted by the radiation emitting component 110. In some examples, at least a portion of the conduit 106 is coated in a biocompatible material for insertion into a cornea. The radiation emitting component 110 can be partially or entirely embedded within the membrane 108. Alternatively, the radiation emitting component 110 is secured to a surface of the membrane without being embedded, e.g., with glue, heat adhesion, soldering, or so forth. In yet another possible embodiment the membrane is not physically connected to the radiation emitting component and the radiation emitting component is either inside or outside the corneal pocket.

The membrane 108 carries the radiation emitting component 110. It should be appreciated, however, that a membrane can alternatively be inserted in a cornea without the radiation emitting component being inserted in the cornea. In some examples, the membrane is constructed from a material or materials selected to absorb radiation emitted by the radiation emitting component 110 that encounters (i.e., propagates towards) the membrane 108 (e.g., propagation towards the front surface 112 of the membrane 108). In some examples, the membrane is constructed from a material or materials selected to reflect radiation emitted by the radiation emitting component 110 that encounters (i.e., propagates towards) the front surface 112 of the membrane 108. For example, the front surface 112 itself can be reflective or absorptive at the wavelength or wavelengths of radiation emitted by the radiation emitting component 110. This can reduce or prevent unwanted exposure of corneal tissue disposed posterior to the posteriorsurface 114 of the membrane 108 to radiation emitted by the radiation emitting component 110. In alternative examples, the membrane is at least partially transparent and/or translucent to radiation emitted by the radiation emitting component 110.

In some examples, the membrane 108 is sized and shaped to fit in a corneal pocket and/or to reduce or prevent radiation exposure to a particular portion of the eye. For example, the membrane 108 can be a round or oval disc shape. Other shapes, including irregular shapes, and membranes having variable thickness, can also be suitable for certain patients or procedures.

In some examples, the membrane 108 is constructed of a biocompatible material or materials that is/are reversibly deformable. That is, the membrane 108 has an undeformed configuration (e.g., as shown in FIG. 1) and a deformed configuration, (e.g., as shown in FIGS. 3 and 4), the membrane being able to return to the undeformed configuration after being deformed. In the deformed configuration, the membrane 108 can assume any desirable configuration, e.g., compressed, rolled, folded (e.g., the second configuration FIG. 3), everted into a U or C-shaped profile, or similar thereto (e.g., FIG. 4). In some examples the membrane 108 is deformed such that the edge 122 of the membrane 108 does not contact another portion of the membrane 108 (e.g., the third configuration of the membrane 108 shown in FIG. 4).

The front surface 112 (and the rear surface 114) of the membrane 108 can have a maximum width w₁ (FIG. 1) when the membrane 108 is in the undeformed configuration. In some examples, the membrane 108 is reversibly deformable such that it can be inserted in the deformed configuration through a corneal incision having a width that is less than w₁, e.g., three fourths, one half, or less, the width w₁.

A practitioner can be provided with the membrane 108 as a separate component from the radiation emitting component 110 and the conduit 106. Alternatively, the membrane is provided to the practitioner already coupled to the radiation emitting component and/or the conduit 106.

FIG. 5 is a schematic side view of a portion 130 of a human eye showing a corneal pocket 132. FIG. 6 is a schematic top view of the portion of the human eye of FIG. 5.

With reference to FIGS. 5-6, the portion 130 of a human eye includes a cornea 134 and an anterior chamber 136. The cornea 134 has a posterior boundary 138 and an anterior boundary 140.

The pocket 132 can be formed by any suitable manner known in the art, e.g., manually, with a femtosecond laser or a mechanical corneal pocket maker. The inventor has previously disclosed systems and methods for making corneal pockets as set forth in, e.g., U.S. Pat. No. 7,901,421, the disclosures of which are incorporated by reference herein in their entirety.

In some examples the pocket 132 is formed between adjacent layers of corneal tissue without excising any tissue. In other examples, a portion of corneal tissue is excised from the pocket 132 prior to insertion of the device 102 (FIG. 1). In the example shown in FIGS. 5-6, the pocket is formed by first making an incision 142 in the anterior surface of the cornea. The incision 142 has a width w₂. In some examples the width w₂ is less than the width w₁ (FIG. 1), and the device 102 (FIG. 1) is deformed such that it can fit through the incision 142 for implantation in the corneal pocket 132 without tearing tissue around the incision 142 or enlarging the incision 142.

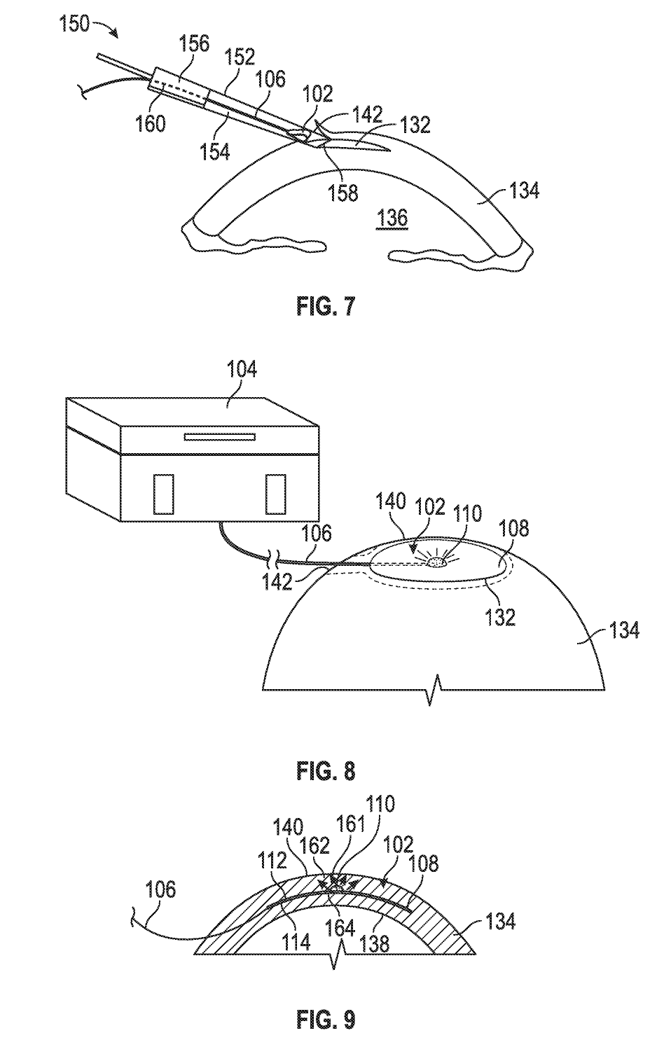

FIG. 7 is a schematic perspective view of the corneal cross-linking device 102 of FIG. 1 disposed in an implantation mechanism 150 for embedding the device 102 in a pocket formed in the cornea of an eye. The corneal pocket 132, the cornea 134, and the anterior chamber 136 of the eye are as described above. In addition, the device 102 is connected to the conduit 106 as described above. The device 102 is shown in a deformed configuration inside the implantation mechanism 150.

The device 102 is implanted in the corneal pocket 132 by any suitable means, e.g., with forceps. In the example shown in FIG. 7 an implantation mechanism 150 is used to implant the device 102 in the corneal pocket 132. The implantation mechanism 150 includes a hollow member 152 having a deformation chamber 154. The implantation mechanism 150 also includes an axial pusher 156. One or more deformation members disposed in the deformation chamber 154 are configured to deform the device 102 as it passes through the deformation chamber 154, urged (through physical contact and/or air pressure differential) by axial movement through the deformation chamber 154 of the axial pusher 156 behind the device 102.

In some examples, the shape of the interior wall of the deformation chamber 154 causes the device 102 to deform into the desired configuration upon its exit from the implantation mechanism 150 at the tip 158 of the deformation chamber 154, the tip being inserted into the corneal pocket 132 via the incision 142.

In the example shown in FIG. 7 an axially aligned or approximately axially aligned bore 160 is disposed through the axial pusher 156 to accommodate the conduit 106. In other examples, the conduit 106 passes along a side of the axial pusher, or an axial pusher is not used and a device 102 is passed through the deformation chamber 154 by other means, e.g., by guiding the conduit 106 by hand or with a grasping tool.

Corneal implant delivery systems employing deformation chambers were previously disclosed by the inventor in, e.g., U.S. Pat. No. 8,029,515, the disclosures of which are incorporated herein by reference in their entirety. It should be appreciated that the device 102 can be implanted in a cornea using the conical implant delivery systems disclosed in the referenced U.S. Pat. No. 8,029,515.

FIG. 8 is a schematic perspective view of the system of FIG. 1, the device of FIG. 1 being disposed in a conical pocket. FIG. 9 is a schematic cross-sectional view of the device of FIG. 1 disposed in a corneal pocket.

With reference to FIGS. 8-9, a cornea 134 having a posterior boundary 138 (FIG. 9), an anterior boundary 140, and a corneal pocket 132 is shown, as described above. A device 102, has been implanted in the corneal pocket 132, the device 102 including the membrane 108 and the radiation emitting component 110, the membrane including the front surface 112 and the rear surface 114, as described above. Also included are the radiation generator 104 and the conduit 106 as described above.

In FIGS. 8-9, the device 102 has been at least partially returned to its undeformed configuration (as shown in FIG. 1) within the corneal pocket 132. In this example at least the membrane 108 portion of the device 102 has been at least partially returned to its undeformed configuration (as shown in FIG. 1) following implantation into the corneal pocket 132 in a deformed configuration (see FIG. 7). To achieve the undeformed or substantially undeformed location in situ, the membrane 108 can be spread out within the conical pocket 132, e.g., with a blunt spatula or other suitable tool.

With reference to FIG. 9, radiation, indicated by the arrows 161, is controllably emitted by the radiation emitting component 110. In this example, the radiation emitting component 110 is disposed on the front surface 112 of the membrane 108. To the extent radiation emitted by the radiation emitting component 110 propagates towards the front surface 112, it is partially or completely reflected by the membrane 108 (e.g., at the front surface 112), thereby reducing or preventing the transmission of radiation to portions of the eye situated behind (i.e., towards the posterior boundary 138 of the cornea) the membrane 108. Meanwhile the radiation 161 is transmitted through desired portions of the cornea situated in front of (i.e., towards the anterior boundary 140 of the cornea) the membrane 108, enabling that radiation to activate a photosensitizer (e.g., riboflavin) present in the corneal tissue. In this manner, the cornea is essentially divided into two regions, a first region 162 disposed anterior to the membrane 108 and through which radiation propagates, and a second region 164 disposed posterior to the membrane 108 and through which radiation is prevented or hindered from propagating by the membrane 108.

Of course, it should be appreciated that, in other examples, modifications to the orientation of the radiation emitting component 110 relative to the membrane 108, and modifications of the orientation of the device 102 when implanted in the cornea (e.g., flipped or angled from what is shown in FIG. 9) will define different regions within the cornea that are irradiated or shielded from radiation.

The practitioner is provided great flexibility in selecting which region or regions of the cornea to irradiate and which region or regions to shield or partially shield from radiation through selection of one or more of: the location and orientation of the corneal pocket; the size shape, and reflective properties of the membrane, the location and type (e.g., singular, plurality), and radiation emitting characteristics (e.g., direction of radiation propagation) of the radiation emitting component, and the placement (e.g., orientation, degree of deformation) of the device 102 within the corneal pocket.

Referring again to FIGS. 8-9, following irradiation of corneal tissue by the radiation emitting component 110, the device 102 is removed from the cornea. Removal of the device 102 can be accomplished by any suitable means, e.g., with forceps or by retracting the device 102 back through an implantation mechanism (e.g., by pulling or drawing the device 102 into the deformation chamber 154 of the implantation mechanism 150 shown in FIG. 7). Thus, it should be appreciated that the device 102 can be removed from the cornea in either a deformed or undeformed configuration. Likewise, the reversible deformability of the device 102 can enable the device for single use and disposal or alternatively repeat use (following proper sterilization).

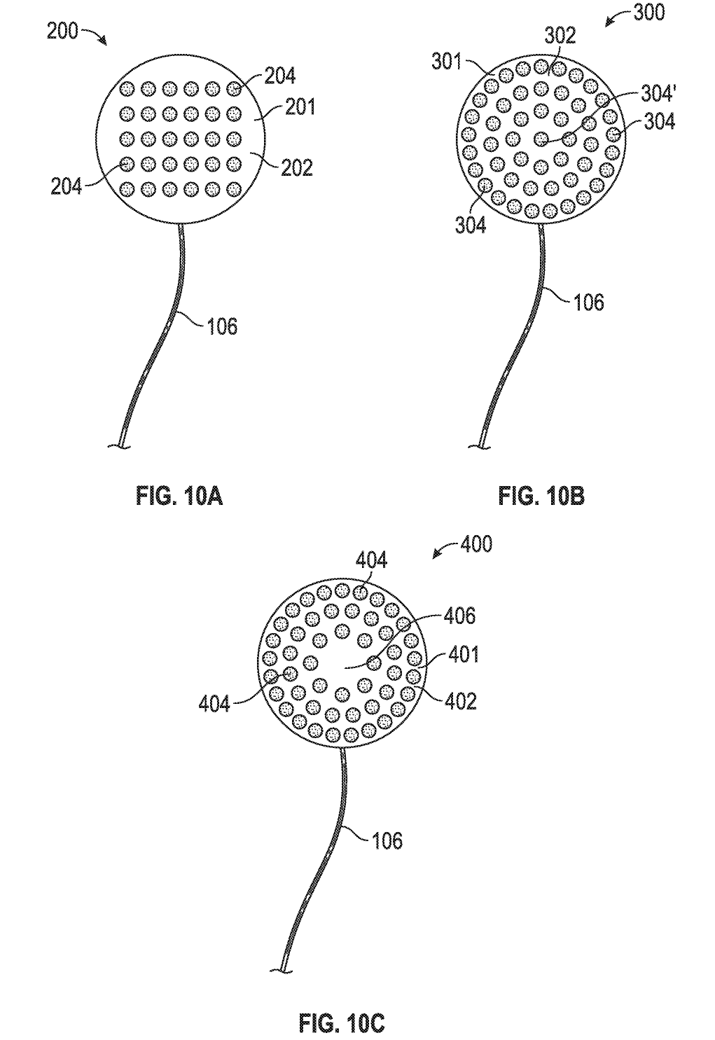

FIG. 10A is a further example of a device 200 for performing cross-linking of corneal tissue in accordance with the present disclosure. FIG. 10B is yet a further example of a device 300 for performing cross-linking of corneal tissue in accordance with the present disclosure. FIG. 10C is yet a further example of a device 400 for performing cross-linking of corneal tissue in accordance with the present disclosure. In each of FIGS. 10A, 10B and 10C, the conduit 106 is shown, as described above.

With reference to FIG. 10A, on the front surface 201 of the membrane 202, a radiation emitting component is disposed consisting of a plurality of radiation emitting elements 204 arranged in a two dimensional rectangular array having a plurality of rows and a plurality of columns. In some examples the radiation emitting elements 204 are LEDs.

With reference to FIG. 10B, on the front surface 301 of the membrane 302, a radiation emitting component is disposed consisting of a plurality of radiation emitting elements 304 arranged in concentric rings, including a single radiation emitting element 304′ disposed at center of the membrane 302. In some examples the radiation emitting elements (304, 304′) are LEDs.

With reference to FIG. 10C, on the front surface 401 of the membrane 402, a radiation emitting component is disposed consisting of a plurality of radiation emitting elements 404 arranged in concentric rings, and without a radiation emitting element disposed in the center 406 of the membrane 402. In some examples the radiation emitting elements 404 are LEDs.

It should be appreciated that the membrane can be provided with additional arrangements of radiation emitting elements, e.g., LEDs. The LEDs can be secured to the surface of the membrane. Alternatively, the LEDs can be partially or entirely embedded in the membrane. In some examples, the membrane comprises a light emitting display, such as an LCD screen.

The plurality of LEDs (or other radiation emitters) can be controllable, e.g., with a computer operating application-specific software that sends electronic signals via the conduit 106 causing the LEDs to be switched on and off in a selected patient-specific pattern and sequence. The type of radiation (e.g., the wavelength), and the intensity of the radiation emitted can also be controllable and can vary from LED to LED. By controlling the characteristics of the radiation being emitted from the arrangement of LEDs, the practitioner can control radiation exposure to different parts of the cornea, enabling precise cross-linking patterns according to what is therapeutically desirable for the patient. In addition to treating degenerative diseases such as keratoconus, controlled radiation emission within the cornea in this manner can also be used to correct refractive errors in healthy corneas, such as myopia, hyperopia, presbyopia and astigmatism or some combination of these refractive errors by strengthening tissue via cross-linking in specific locations or areas.

A method for cross-linking corneal tissue in accordance with the present disclosure includes: making an incision in the cornea; making a corneal pocket accessible from the incision; introducing a photosensitizer (e.g., with a syringe) into the corneal pocket and allowing sufficient absorption into corneal tissue; reversibly deforming a device having a membrane and a radiation emitting component; implanting the device in the corneal pocket via the incision; at least partially reversing the deformation of the implanted device within the corneal pocket; activating the radiation emitting component to cause the radiation emitting component to emit radiation; and removing the implanted device from the corneal pocket. In some examples, the method includes an additional step of sealing the incision after removal of the device, e.g., with glue, sutures, or so forth.

In some examples of the method, the incision has a width that is smaller than a largest width of the membrane. In some examples the corneal pocket is made approximately round in shape, having a diameter of about 3 mm to about 12.5 mm, and a depth from the anterior corneal surface between about 80 μm from the anterior boundary (i.e., the epithelial layer) of the cornea to about 20 μm from the posterior boundary (i.e. the endothelial layer) of the cornea. Depths outside of these ranges may also be suitable.

In some examples of the method, the photosensitizer is a riboflavin solution, the solution having a riboflavin concentration from about 0.01% to about 0.3%, with a volume of solution introduced into the pocket in a range from 10 μL to about 200 μL. In some examples, the solution is allowed to diffuse into corneal tissue for a duration in a range from about five minutes to about sixty minutes. Concentration, volumes and durations outside of these ranges may also be suitable.

In some alternative examples of the method, the device is implanted in the pocket prior to introducing the photosensitizer to the pocket. In these examples, the membrane may act inhibit diffusion of the photosensitizer to certain parts of the cornea.

In some examples of the method, the device is deformed prior to implantation in the corneal pocket such that it can fit through an incision that is smaller (e.g., less than three fourths or less than half) the device's largest width in an undeformed configuration. In some examples, the device is deformed and/or implanted into the corneal pocket using an implantation mechanism. The implantation mechanism may optionally include a deformation chamber, one or more deformation members, and/or an axial pusher.

In some examples of the method, the membrane has a maximum width in an undeformed configuration in a range from about 3 mm to about 13 mm to encompass the range of treatment areas that would be clinically useful. Dimensions outside of this range may also be suitable. In some examples, the membrane includes at least one reflective element, such that the membrane at least partially reflects the radiation emitted by the radiation emitting component, and is manufactured from one or more of polymeric films, metallic films, or foil. In some examples the membrane includes a polymer on which a reflective metal is bonded.

In some examples of the method, the at least partially reversing the deformation of the device within the corneal pocket is achieved by flattening the membrane, e.g., with a spatula and then removing any device used for flattening from the corneal pocket. In some examples, the membrane is configured to automatically revert to or towards its undeformed configuration upon its release into the corneal pocket.

In some examples of the method, the radiation emitting component emits UV light in a continuous or non-continuous manner for a duration from about five minutes to about sixty minutes at a wavelength in a range from about 365 μm to about 380 μm and a power in a range from about 1 mW/cm² to about 10 mW/cm². Wavelengths and durations outside of these ranges may also be suitable.

In some examples of the method, following irradiation, the device is deformed prior to or during its removal from the corneal pocket, e.g., using the implantation mechanism.

In some examples of the method in which cross-linking is indicated near the anterior surface of the cornea, the epithelial layer of the cornea is removed, and the photosensitizer solution is introduced to the deepithelized surface of the cornea instead, or in addition to, introduction of the photosensitizer solution via the corneal pocket. It should be noted that introducing a photosensitizer solution via corneal pocket may be less painful than removing the epithelium.

While the above is a complete description of certain embodiments of the invention, various alternatives, modifications, and equivalents may be used. Therefore, the above description should not be taken as limiting the scope of the invention which is defined by the appended claims.

1. A device for performing cross-linking of corneal tissue, the device comprising:
a membrane; and
a radiation emitting component, the membrane being configured to be removably embedded in a cornea.

2. The device of claim 1, wherein the membrane is reversibly deformable.

3. The device of claim 1, wherein the membrane comprises a reflective element, and wherein the membrane at least partially reflects radiation emitted by the radiation emitting component.

4. The device of claim 1, wherein the membrane comprises an undeformed configuration and a deformed configuration, and wherein the membrane is configured to return to the undeformed configuration from the deformed configuration inside a corneal pocket.

5. The device of claim 1, wherein the radiation emitting component is configured to emit UV radiation.

6. The device of claim 5, wherein the membrane is configured to reflect UV radiation.

7. The device of claim 5, wherein the UV radiation is selected to activate a photosensitizer agent present in the cornea.

8. The device of claim 7, wherein the photosensitizer agent comprises riboflavin.

9. The device of claim 1, wherein the radiation emitting component is connected to a conduit, the conduit configured to transmit optical signals from a radiation generator to the radiation emitting component.

10. The device of claim 9, wherein a portion of the conduit is embedded in the membrane.

11. The device of claim 1, wherein the radiation emitting component comprises a plurality of radiation emitting elements.

12. The device of claim 11, wherein the radiation emitting elements form an array at least partially embedded in the membrane.

13. The device of claim 12, wherein the controller is configured to control a pattern of radiation emitted by the array when the device is embedded in a cornea.

14. The device of claim 12, wherein the array is rectangular, and wherein the array comprises at least one row of radiation emitting elements.

15. The device of claim 12, wherein the array comprises at least one ring of radiation emitting elements.

16-30. (canceled)
